(19) United States
(12) Patent Application Publication
(10) Pub. No.: US 2023/0170065 A1
(43) Pub. Date: Jun. 1, 2023

(54) TREATMENT RECOMMENDATION

(71) Applicant: Arine, Inc., San Francisco, CA (US)

(72) Inventors: David De Vries, San Francisco, CA (US); Eunice Kim, Los Angeles, CA (US); Yoona Kim, San Francisco, CA (US); Amit Klein, Danville, CA (US); Jeffrey Rose, San Francisco, CA (US); Penjit Moorhead, San Mateo, CA (US)

(21) Appl. No.: 17/921,928

(22) PCT Filed: Apr. 30, 2021
(86) PCT No.: PCT/US2021/030258

§ 371 Date: Oct. 27, 2022

Related U.S. Application Data
(60) Provisional application No. 63/018,493, filed on Apr. 30, 2020.

(51) Int. Cl. G16H 20/00; G16H 50/30; G16H 10/20

(52) U.S. Cl. CPC G16H 20/00; G16H 10/20; G16H 50/30

(57) ABSTRACT

In one aspect, data characterizing healthcare information associated with a patient can be received. A health outcome evaluation can be determined for the patient based on the received healthcare information data. A risk prediction for the patient can be determined based on the determined health outcome evaluation. A treatment recommendation for the patient can be determined based on the determined risk prediction, and the treatment recommendation can be provided.

### CROSS-REFERENCE TO RELATED APPLICATIONS

This application claims priority under 35 U.S.C. § 119(e) to U.S. Provisional Pat. Application Number 63/018,493 filed Apr. 30, 2020, the entire contents of which is hereby expressly incorporated by reference herein.

### TECHNICAL FIELD

The subject matter described herein relates to providing recommendations for treatment of a patient, for example, recommendations for a complete, optimal patient care plan.

### BACKGROUND

Providing comprehensive treatment information to patients can require large amounts of data to be assimilated from many disparate sources. The data sources may not include the most current patient health data, expert clinical guidance, and holistic breadth needed to properly inform patients about the most optimal course of treatment (e.g., care plan) to best manage chronic conditions, to improve health, to improve wellbeing, and the like. Treatment plan errors, which can include incorrect prescription writing and taking, medication access issues, and adverse drug events due to unsafe combinations of medications, and the like. Indeed, the incidence and risk of non-optimized treatment plans and can be exponentially increased when patients are prescribed, for example, four or more medications or when patients are prescribed medications and other treatments from more than one provider. Other contributors of drug-related problems can include insufficient information transfer between providers and lack of disclosure from patients on other over-the-counter and herbal medications they are taking.

Patient interventions intended to avoid these negative consequences can be provided at predefined time points as a pharmacist or physician-performed in person interview. However, these interventions only take into account each patient’s health status at a cross-sectional point in time without considering past or future health risks. In some instances, these existing solutions do not consider the patient’s past or future health risks, and instead intervene at predefined time points that are independent from clinical observations and behavioral observations of the patent, rather than the point at which the patient needs an intervention due to increased health risks. Such limitations can lead to patients having limited access to highly needed treatment education and counseling over the course of their care, thereby exacerbating a sustained rate of medication errors.

### SUMMARY OF THE INVENTION

Methods and systems for treatment recommendations are provided. Related apparatus, techniques, and articles are also described.

In one aspect, data characterizing healthcare information associated with a patient can be received. A health outcome evaluation can be determined for the patient based on the received healthcare information data. A risk prediction for the patient can be determined based on the determined health outcome evaluation. A treatment recommendation for the patient can be determined based on the determined risk prediction, and the treatment recommendation can be provided.

One or more of the following features can be included in any feasible combination. For example, the determining of the health outcome evaluation can include comparing the received healthcare information data to healthcare data characterizing a predetermined set of healthcare parameters for an aggregated population of patients, determining a deficiency in the received healthcare information data based on the predetermined set of healthcare parameters, generating questionnaire data that characterizes at least one question based on the determined deficiency, providing the questionnaire data to a client device of the patient, and receiving, from a client device, answer data characterizing at least one answer to the at least one question characterized by the questionnaire data, and the health outcome evaluation can be based on the answer data. For example, the generating of the questionnaire data can include querying a questionnaire rules engine for the at least one question based on the determined deficiency, the questionnaire rules engine configured to generate the at least one question, the questionnaire rules engine modified by a questionnaire predictive model that identifies a predictor variable based on the received healthcare information data and revises the questionnaire rules engine based on the identified predictor variables, and receiving the at least one question from the questionnaire rules engine for inclusion in the questionnaire data. For example, a clinical patient profile can be determined for the patient based on the received healthcare information data and the determined health outcome evaluation, and the clinical patient profile can characterize an attribute of the patient. For example, a provider profile can be determined for a provider of healthcare servers to the patient based on the received healthcare data, and the provider profile can characterize an attribute of the provider. For example, the determining of the risk prediction for the patient can include executing a risk prediction model for a risk factor that predicts a likelihood of a negative health outcome, the risk prediction model trained for providing the risk factor in response to the querying based on historical patient risk data. For example, the determining of the treatment recommendation can include querying a treatment recommendation rules engine for a recommendation parameter based on at least one of the determined risk factor, the health outcome evaluation, and/or the received healthcare information data, the querying including execution of a recommendation rule by the treatment recommendation rules engine, and generating a recommendation string that characterizes the recommendation parameter. For example, the treatment recommendation rules engine can be modified by a predictive model that identifies a predictor variable characterizing a likelihood of success of an intervention characterized by the treatment recommendation, the identifying based on received feedback data that indicates a level of success of the intervention, determines a modification to the recommendation rule based on the identified predictor variable, and modifies the recommendation rule based on the determined modification. For example, the providing of the treatment recommendation can include transmitting the recommendation string for presentation on a graphical user interface of a client device. For example, the treatment recommendation rules engine can be modified by a recommendation predictive model that identifies a predictor variable characterizing a pattern in adherence to interventions suggested by the treatment recommendation based on the received healthcare information data and modifies a rule of the treatment recommendation rules engine based on the identification. For example, the determining of the risk prediction for the patient includes determining a clinical risk parameter characterizing a level of clinical risk based on the determined health outcome evaluation, determining a social risk parameter characterizing a level of social risk based on the determined health outcome evaluation, and determining a behavioral risk parameter characterizing a level of behavioral risk based on the determined health outcome evaluation. For example, one or more of the clinical risk parameter, the social risk parameter, and the behavioral risk parameter can be dynamically updated based on received feedback data characterizing the patient.

In another aspect, a system is provided and include at least one data processor and memory storing instructions configured to cause the at least one data processor to perform operations described herein. The operations can include receiving data characterizing healthcare information associated with a patient, determining a health outcome evaluation for the patient based on the received healthcare information data, determining a risk prediction for the patient based on the determined health outcome evaluation, determining a treatment recommendation for the patient based on the determined risk prediction, and providing the treatment recommendation.

One or more of the following features can be included in any feasible combination. For example, the determining of the health outcome evaluation can include comparing the received healthcare information data to healthcare data characterizing a predetermined set of healthcare parameters for an aggregated population of patients, determining a deficiency in the received healthcare information data based on the predetermined set of healthcare parameters, generating questionnaire data that characterizes at least one question based on the determined deficiency, providing the questionnaire data to a client device of the patient, and receiving, from a client device, answer data characterizing at least one answer to the at least one question characterized by the questionnaire data; and the health outcome evaluation can be based on the answer data. For example, the generating of the questionnaire data can include querying a questionnaire rules engine for the at least one question based on the determined deficiency, the questionnaire rules engine configured to generate the at least one question, the questionnaire rules engine modified by a questionnaire predictive model that identifies a predictor variable based on the received healthcare information data and revises the questionnaire rules engine based on the identified predictor variables, and receiving the at least one question from the questionnaire rules engine for inclusion in the questionnaire data. For example, the determining of the risk prediction for the patient can include executing a risk prediction model for a risk factor that predicts a likelihood of a negative health outcome, the risk prediction model trained for providing the risk factor in response to the querying based on historical patient risk data. For example, the determining of the treatment recommendation can include querying a treatment recommendation rules engine for a recommendation parameter based on at least one of the determined risk factor, the health outcome evaluation, and/or the received healthcare information data, the querying including execution of a recommendation rule by the treatment recommendation rules engine, and generating a recommendation string that characterizes the recommendation parameter. For example, the treatment recommendation rules engine can be modified by a predictive model that identifies a predictor variable characterizing a likelihood of success of an intervention characterized by the treatment recommendation, the identifying based on received feedback data that indicates a level of success of the intervention, determines a modification to the recommendation rule based on the identified predictor variable, and modifies the recommendation rule based on the determined modification. For example, the treatment recommendation rules engine can be modified by a recommendation predictive model that identifies a predictor variable characterizing a pattern in adherence to interventions suggested by the treatment recommendation based on the received healthcare information data and modifies a rule of the treatment recommendation rules engine based on the identification.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The embodiments described above will be more fully understood from the following detailed description taken in conjunction with the accompanying drawings. The drawings are not intended to be drawn to scale. For purposes of clarity, not every component may be labeled in every drawing. In the drawings:

FIG. 1 is a process flow diagram illustrating an example process of some implementations of the current subject matter that can provide for treatment recommendations;

FIG. 2 is a system diagram illustrating an example system of some implementations of the current subject matter that can provide for treatment recommendations; and

FIG. 3 is a data flow diagram illustrating the transfer of data between the system components illustrated in FIG. 2.

Like reference symbols in the various drawings indicate like elements.

### DETAILED DESCRIPTION

Some current processes for providing information regarding a patient’s care plan, medication treatment and administration can be overly narrow (e.g., adherence), laborious (e.g., call centers), and can have limited scale and lead to minimal improvement. Patient interventions can be provided at predefined time points as a pharmacist or physician-performed in person interview, and can take into account each patient’s health status at a cross-sectional point in time without considering past or future health risks. These issues can lead to patients having limited access to highly needed treatment education and counseling over the course of their care, thereby exacerbating a sustained rate of medication errors. Some existing approaches and software products for providing recommendations for treatment of conditions and administration of medications have been shown to have limited clinical impact, highlighting the need for better strategies and platforms.

Some implementations of the current subject matter can provide an improved approach to providing treatment information as described herein. Some implementations of the current subject matter can advantageously intake and analyze multiple, disparate sources of data to generate prioritized treatment recommendations for patients by execution of a dynamic rules engine, and the methodology for prioritization of the treatment recommendations can be determined at least by training the dynamic rules engine with historical data that associates successful treatment outcomes with various treatment recommendations. As such, robust treatment information and tailored treatment recommendations for the patient can be curated and efficiently disseminated to address gaps in care and non-optimized treatments, adjust existing care plans, avoid medication and prescription errors, avoid adverse drug events, and avoid and address healthcare and medication access issues. Accordingly, treatment recommendations can be provided for each patient’s needs at the right place and right time in their care journey.

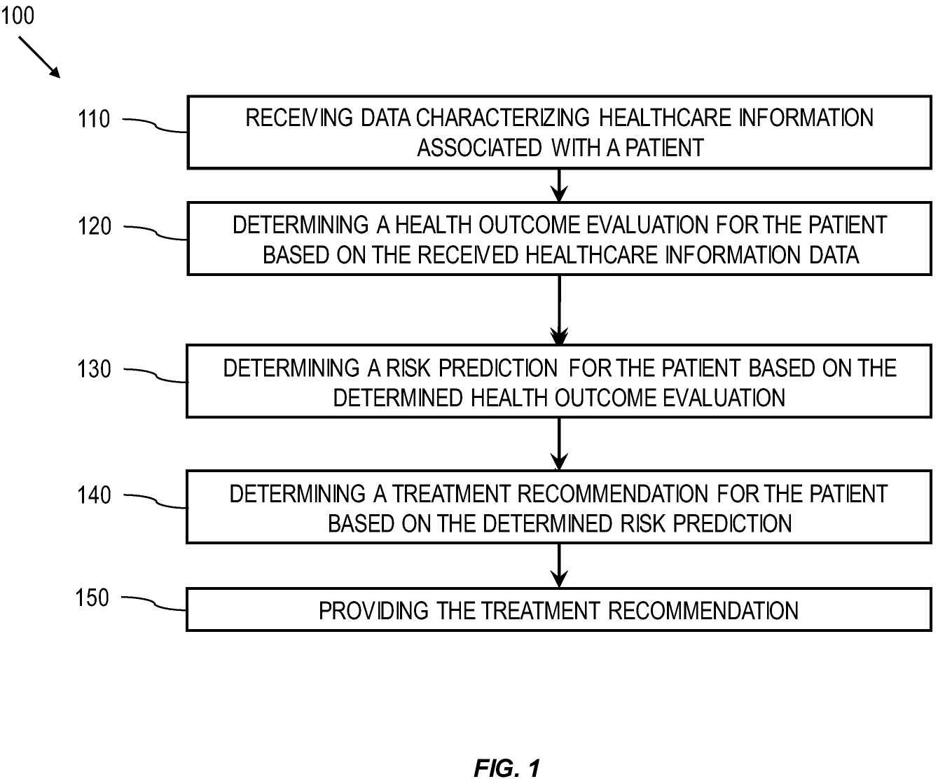

FIG. 1 is a flowchart illustrating an example process 100 for providing treatment recommendations for a patient according to subject matter described herein. At operation 110, data characterizing healthcare information associated with a patient can be received. The received healthcare information data can include data characterizing the patient’s prescription claims, medical insurance claims, healthcare utilization, diagnoses, behavior, demographics, prior authorizations, electronic medical records (e.g., lab data and medical chart data), and adherence to prescribed treatment plans. In some implementations, the received healthcare information data can include data obtained from wearable devices used for patient monitoring, health applications or software, answers to patient questionnaires provided by the patient, answers to risk assessments provided by the patient, patient geolocation data, and lab and/or genomic data characterizing patient attributes. In some implementations, the received healthcare information data can also include data characterizing insurance claims billing, electronic health records, answers to algorithmically-derived health questions, case management, patient health plans, and drug coverage data. Additional data associated with a patient’s medical status, such as medical data received from healthcare providers interfacing with patients in inpatient and/or outpatient settings, can also be included.

In some implementations, the healthcare information data can be received from one or more client devices of the patient directly. The one or more client devices of the patient can include a platform interface of a mobile device of the patient (e.g., a web page or an application executable on the mobile device), a platform interface of a personal computer of the patient (e.g., a web page or an application executable on the personal computer), a wearable device configured to measure health parameters and/or biomarkers of the patient and to transmit data characterizing the measured health parameters and/or biomarkers to the platform, and a portable medical device configured to measure the health parameters and/or the biomarkers of the patient and to transmit data characterizing the measured health parameters and/or biomarkers.

In some implementations, the healthcare information data can be received from devices of healthcare providers and/or clinicians. Such devices can include mobile devices, personal computers, and/or medical devices of the providers and/or clinicians that can include the same or similar capabilities as those of the patients described above. For example, in some implementations, data provided by a provider can include information regarding their patients, responses to recommendations sent for their patients, including whether the recommendation was implemented and the clinical rationale. Other provider reported data can include clinical questions answered by the patient during a provider intervention, patient history as reported to providers, lab data and other vitals available to the provider, medications prescribed for specific patient types and diagnoses, patient information such as vital signs, latest visit dates and purpose, information about their diagnoses or medications, or other healthcare outcomes evaluation data related to a patient.

In some implementations, the received healthcare information data can also include information about a healthcare provider such as a physician. For example, the received healthcare information data can include a prescribing pattern of the provider, data characterizing a location of the provider, data characterizing provider associations with other providers, data characterizing provider associations with patients, demographic data characterizing demographic attributes of the provider, and data characterizing past interventions in patient health made by the provider. In some implementations, the received healthcare information data can also include data characterizing a specialty of the provider, a prescribing history of the provider, the provider implementation of previously-received treatment recommendations by the provider (explained in further detail below), an education level of the provider, previous healthcare interventions made by the provider, quality scores characterizing the provider’s performance, a number of patients associated with the provider (e.g., provider panel size, referral networks of the provider, insurance companies plans/accepted by the provider, payment data associated with the provider, and more.)

In some implementations, the received healthcare information data can also include drug data from such external databases as a drug information database. The drug data can characterize information on medication name, dosage, provenance, appearance, known drug-to-drug interactions and manufacturing information including known indications it is used for and published side effects. In some implementations, the received data can also include data such external databases as a prescription history database and a medical history database, which can be licensed from a third party, and can contain historical information on the patient’s medications, medical history and healthcare utilization that other data sources coupled to the software platform may not have access to. For example, in the case of a patient switching health plans, a third party may have access to the information for the patient when they were a member of PLAN A, and the software platform can be configured to acquire the ongoing customer data feed from PLAN B.

In some implementations, the received data can include healthcare data from multiple sources that characterizes health information for a patient population. The healthcare data can include medical claims data, pharmacy claims data, risk stratification data, quality of care data, electronic medical record data, lab value data, utilization data, wearable and diagnostic device data, socioeconomic data, program eligibility data and demographic data, pharmacogenomic data, clinical trial data, social network data, e-prescribing data, electronic prior authorization data, other digital device data from data sources coupled to the software platform described herein. The source(s) of the healthcare data can include users or beneficiaries of the software platform, which can include patients, healthcare providers/clinicians, health insurance companies, pharmacy benefit managers, local and state government entities, care management companies, hospitals and health systems, medical groups, retail pharmacies, pharmaceutical companies, accountable care organization or other enterprise healthcare companies, as well as patients or members of a health insurance plan. The data from each of these sources can be combined (“aggregated data”) to form a single dataset, as discussed in further detail below. The platform can intake and aggregate data from an infinite number of sources.

The data can be received from the aforementioned data sources via an ongoing (real time) or regularly scheduled data feed, or ad-hoc. The received data can be supplemented with additional data gathered directly from patients and providers over time.

All received data can be stored using schema-based data storage (e.g., RDS, PostGres), as well as document-based data storage (e.g., DynamoDB) which can allow the software platform to store more complex data structures and schema than flat relational data. This can allow the software platform to have flexibility in terms of being able to store any kind or type of data. The data structure format can allow for a single clinical profile to be deepened with any new data type or format in an ongoing basis regardless of frequency or data structure/format.

At 120, a health outcome evaluation for the patient can be determined based on the received healthcare information data. Datasets evaluated as part of the health outcome evaluation include survey responses, medical claims data, pharmacy claims data, lab data, other questionnaire data, patient-reported data, provider-reported data, other cost data, third party data sources (e.g. prescription data from SureScripts, risk or credit data from LexisNexis) and electronic health records. The evaluated data can include historical data on the patient and their care plan, treatment patterns and health history up until present time.

In some implementations, the health outcome evaluation can include metrics that can be determined based on the received healthcare information data. The determined metrics can include utilization patterns (e.g., hospitalizations, emergency department visits, clinic visits), clinical values (e.g., A1c for a diabetes patient, blood pressure for a hypertensive patient), healthcare spending patterns (e.g., pharmacy claims cost, medical claims cost, out of pocket costs), medication information (e.g., drug classes, generic, brand, formulary), medication taking patterns, questionnaire responses (e.g., survey response improvement in general anxiety disorder questionnaire, reasons for nonadherence), disease profile (e.g., diagnoses, duration, utilization by disease), provider actions (e.g., prescribing behaviors, quality metrics, interventions), patient demographic and engagement profile. The metrics can be determined based on data characterizing patient demographics, medication refills, diagnoses, inpatient/outpatient/ emergency department/clinic visits, provider demographics, laboratory demographics, and/or pricing information, which can be normalized, transformed, and/or aggregated from the received healthcare information data.

In some implementations, the metrics can be determined based on data characterizing provider information, such as a National Provider Identifier (NPI) record. In some implementations, the metrics can be determined based on data characterizing such drug information as National Drug Code (NDC) to drug mapping, drug indications, drug-drug interactions, mapping of drug groups, and/or drug images. In some implementations, the metrics can be determined based on data characterizing such diagnostic information as ICD10/CPT code to drug group mapping and diagnostic group mapping. In some implementations, the metrics can be determined based on data characterizing questionnaires that are tailored to a patient based on the received healthcare information data (as explained in further detail below). In some implementations, the metrics can be determined based on subsets of the received healthcare information data that is reported directly by the patient via their client device, a wearable device, and/or a medical device of the patient. Such data subsets can include data characterizing medications taken by the patient, medication-taking behaviors of the patient, medication-related questions/needs, health status, and patient engagement. In some implementations, the metrics can be determined based on a subset of the received healthcare information data that characterizes treatment decisions made by providers that pertain to a patient or a population of patients with a similar health characteristic.

In some implementations, the health outcome evaluation can be determined based on patient data that is a subset of the received healthcare information data and that characterizes a patient’s demographic, geo-location, socioeconomic, and healthcare engagement attributes. In some implementations, the health outcome evaluation can be determined based on data that is a subset of the received healthcare information data and that characterizes a patient’s medication information, such as drug image, drug group, dose form, route, dosage unit, indications, and prescriber. In some implementations, the health outcome evaluation can be determined based on data that is a subset of the received healthcare information data and that characterizes a patient’s receipt of medications (e.g., dosing regimen, dose per day, monthly prescribing reference (MPR), reasons for taking medications, reasons for discontinuing medications, side effects, drug interactions). In some implementations, the health outcome evaluation can be determined based on patient data that is a subset of the received healthcare information data and that characterizes a disease profile of the patient (e.g., disease duration, diagnosis groups, healthcare utilization, and abnormal lab values). In some implementations, the health outcome evaluation can be determined based on provider data that is a subset of the received healthcare information data and that characterizes provider actions (e.g., prescribing behaviors, quality metrics, intervention types, and intervention frequencies). In some implementations, the health outcome evaluation can be determined based on customizable data flags that are programmed as necessary to achieve actionable evaluations from the received healthcare information data.

In some implementations, the health outcome evaluation can be determined based on data from clinical knowledge databases, which can contain a compendium of treatment guidelines, a compendium of real world evidence and data on treatment regimens and their impact on clinical outcomes, a compendium of quality metrics and best practices, a compendium of clinical knowledge and information related to ideal treatment regimens for patients based on specific characteristics. The health outcome evaluation can be further updated based on data from client-specific databases, which can contain information on the customers’ available programs and services, including details of their benefits and coverage levels, their formulary, their patient or member management programs, their costs, their assistance programs, and their specific preferred treatments and sites of care. In some implementations, the health outcome evaluation can be further updated based on other third-party programs and databases.

In some implementations, the health outcome evaluation can output data characterizing other identified problems and potential issues, such as a detailed understanding of a patient’s adherence to their current treatment regimen as prescribed, their adherence and compliance to the treatment regimen, the risks of their current treatment regimen, missing elements of their treatment regimen such as medications, clinical tests, provider visits, their clinical risk profile and likelihood of clinical events such as hospitalization, dangerous medication combinations including incorrect dosage, combination, or unnecessary prescription and other health behaviors. In some implementations, the health outcome evaluation can output data, based on one or more of the aforementioned sources of data, that characterizes a patient’s healthcare utilization, trends in a patient’s clinical status, trends in a patient’s behaviors, trends in a provider’s prescribing behaviors, trends in healthcare costs, and data characterizing risks faced by the patient.

The health outcome evaluation and these aforementioned data outputs can be determined by health outcome evaluation algorithms that can selectively determine the content for inclusion in the outputted data based on the types/attributes of the aforementioned data/metrics that form the basis of the health outcome evaluation. The health outcome evaluation algorithms can assess the data/metrics discussed above that can form the basis of the health outcome evaluation in the extract-transform-load (ETL) process, and the ETL executes the algorithms on the data/metrics to determine the health outcome evaluation. In some implementations, one or more algorithms can be applied to the received healthcare information data and metrics described above, and a series of tailored questions can be generated, for presentation to the patient, that are configured to address one or more deficiencies in the received data that are detected by the one or more algorithms. For example, the one or more algorithms can analyze the received data by using a questionnaire rules engine which evaluates the data sources received and identifies deficiencies in the data corresponding to the patient that would be critical to drive clinical decisions. These deficiencies can then be analyzed by the questionnaire rules engine, and the questionnaire rules engine can generate questionnaire data that characterizes at least one question to be answered by the patient and/or their caregiver and that is configured to address the deficiencies. For example, if the patient is prescribed metformin, but not regularly taking this medication, a tailored question configured to determine whether the patient is experiencing any side effects to metformin can be generated by the questionnaire rules engine. In another example, if a patient is on Medicaid, or lives in a low income ZIP code area, questions regarding social determinants of health will surface (e.g., transportation barriers, housing barriers, cost barriers, etc.).

In some implementations, the questionnaire data can be provided to patients (and/or their caregivers) via a web interface of a device of the patient/caregiver, in person, or telephonically. The questionnaire data can include questions that can mimic best-in-class clinical interviews performed by pharmacists, nurses, physicians and other qualified healthcare professionals in care settings. In some implementations, the questionnaire data can include questions that can be configured to current and historic medication-taking behaviors, side effects, and patient-reported symptoms, outcomes and such issues with access to care as cost, difficulties making appointments, low literacy/health literacy, educational barriers, transportation difficulties, and the like.

In some implementations, the questionnaire rules engine can determine the questions for inclusion in the questionnaire data based on the received healthcare information data. For example, if the received healthcare information data indicates that the patient lives in a low income ZIP code, is a Medicaid beneficiary, low income subsidy beneficiary, or in other programs identified through eligibility information indicating low income, the questionnaire rules engine can analyze these attributes of the received healthcare data and make the determination that access to care questions should be included in the questionnaire data. In another example, if the received healthcare information data indicates that the patient has received a diabetes diagnosis, the questionnaire rules engine can generate specific questions configured to ascertain additional information related to the patient’s management of their diabetes, such as questions intended to ascertain the patient’s last blood sugar reading, the patient’s eating habits, whether the patient has felt dizzy or lightheaded while taking their medications, whether the patient has experienced any difficulty using their insulin, etc. In another example, if the received healthcare information data indicates that the patient lives in a food desert and has low income, the questionnaire rules engine can generate specific questions configured to ascertain additional information related to the patient’s food security and ability to pay for their medications or doctor visits, whether they need coupons/patient assistance programs, etc. And, in another example, if the received healthcare information data indicates that the patient that has trouble walking, the questionnaire rules engine can generate specific questions configured to ascertain additional information related to their preference to receive mail order medications, transportation assistance for medical appointments, home health support and care management or virtual healthcare etc.

In some implementations, the questionnaire rules engine can compare the received data to aggregated healthcare data that characterizes a predetermined set of healthcare parameters , determine the one or more deficiencies in the received healthcare information data based on the comparison, and determine the questions for inclusion in the questionnaire data based on the determined deficiencies.

In some implementations, one or more algorithms can analyze the received healthcare information data, determine whether the received healthcare information data indicates ongoing negative health behaviors of the patient, and generate the questionnaire based on the analysis to determine further insights about the patient. For example, when a patient is nonadherent to their medications, the one or more algorithms can identify the occurrence of the nonadherence by analyzing the received data and generate, based on the identified nonadherence, one or more questions that are configured to obtain, from the patient, the patient’s reason for nonadherence (e.g., side effects, cost barriers, lack of understanding of importance). Answers to these questions described above can be analyzed to provide further depth and context to patient health status and behaviors. The questionnaire data, which provides insights into patient behaviors otherwise unknown, can be combined with the received data to create an entirely new dataset from which a more comprehensive healthcare evaluation dataset can be determined as explained further below.

In some implementations, the questionnaire rules engine can be continuously improved by the use of predictive modeling techniques. For example, in some implementations, data is collected on whether the patient reports side effects to a particular medication through the questionnaires. A predictive model can be used to evaluate the data and to identify significant predictors of experiencing side effects for a particular populations. The predictive model can modify the rules utilized by the questionnaire rules engine based on the identified significant predictors and thereby can generate tailored questions based on the modified rules that are targeted to the populations characterized by the predictors.

In some implementations, a clinical patient profile for a patient can be determined based on the received healthcare information data and the determined health outcome evaluation. In some implementations, the clinical patient profile can include a graphical user interface that characterizes the attributes of a patient, such as the patient’s healthcare utilization, their current and historical hospitalization and emergency department visits, their current and historical diagnoses, and their current and historical medication usage, as well as gaps in care, non-optimal care plans, demographic information, contact information, lab and other test results, health insurance information, risk assessment information, current and historical clinical questionnaire information, and preferred methods of intervention. The clinical patient profile can also include the patient’s current and past over-the-counter medication and supplement usage, the patient’s care team composition (e.g., an identity of the patient’s primary care provider, specialists, and/or pharmacies, etc.), the patient’s current care and treatment plans, the patient’s lab tests and clinical values, and any recommended care plan changes for the patient.

In some implementations, population-level provider profiles can be determined based on the received healthcare data. The provider profile data includes derived metrics from the health outcome evaluation on the provider demographics, such as location, type of provider, and provider actions, such as prescribing behaviors and interventions. The provider profiles can include an easily interpreted view of healthcare data of a panel of patients associated with a provider, and information about their patients, such as the panel’s current gaps in care, the panel’s healthcare service utilization. The provider profiles can also include data characterizing patient demographic information, such contact information as patient and/or provider phone numbers, email addresses, mailing addresses, fax numbers and other telecom info. In some implementations, the provider profile can also include an overview of the makeup of their patient panel by age, insurance type, location (home address) and other descriptors. In some implementations, such data can be sourced from third party data sources or directly from the provider or patient via data input from telephonic outreach or other communications.

In some implementations, the provider profiles can include health insurance information characterizing insurance plans accepted by the provider as well as the insurance plan of the patient, risk assessment information characterizing the provider’s overall panel risk profile for their patients, the risk profile of the provider, and current and historical clinical quality performance of the provider (such as their performance on specific Healthcare Effectiveness Data and Information Set (HEDIS) quality measures or other metrics used to evaluate their performance, prescribing patterns of the provider, treatment plan patterns of the provider, patient panel information). In some implementations, the provider profile can include data characterizing a number of patients used as the basis for the data characterizing the provider in the provider profile, locations such as home addresses of their patient panel, provider practice locations, the type of insurance carried by their patients, the patient’s average distance from a provider, a number of times the patient was seen by provider during a specific time period, and preferred methods of intervention and communication, such as how the provider prefers to receive information (e.g. email, text, phone call, fax). In some implementations, patient profiles can also be aggregated and assigned to a provider (e.g. Patient A and Patient B are both seen by physician X, and recommendations 1,2,3 are sent to the provider, where 1 and 2 are assigned to patient A and 3 is assigned to patient B).

At 130, a risk prediction for the patient can be determined based on the health outcome evaluation. In some implementations, the risk prediction for the patient can be determined based on the received data, described above, that characterizes the interventions generated from the platform and the resulting outcomes of those interventions. The risk prediction can be determined by predictive multivariable models that analyze one or more aspects of the data included in the health outcome evaluation, such as data on diagnoses, medication history, answers to the tailored questions, clinical variables, the patient’s demographic information, provider data characterizing the prescribers associated with a patient, their locations, prescribing patterns, quality scores, patient outcomes data characterizing past interventions, and more.

As such, the risk prediction models can generate an overall risk prediction for the patient as well as for a risk prediction for each of the clinical, social, and behavioral risk subcategories. Clinical factors can include medical diagnoses, medication regimen, healthcare utilization, other prescribed treatments, over the counter supplements, clinical history, physical measurements, clinical and lab values including vitals, genomic data, validated clinical questionnaire data and more. Social factors include demographics information such as age, gender, race, zip code, occupation, occupational status, education, food security status, housing status, income, health insurance status, health literacy, care access, air and water quality, incarceration status, family composition, caregiver status, marital status, stressors, social support and more. Behavioral factors include smoking, alcohol consumption, physical activity, obesity, diet, sexual health, sleep patterns, medication-taking behaviors and more. As explained in further detail below, the risk prediction for each risk subcategory can be used to associate the risks with the most likely recommendations to reduce those risks, so as to determine the correct recommendations that best address each patient’s unique needs. In some implementations, statistical methods can be utilized to break up the overall risk assessment from these learned models into these subcategories based on the presence of prediction variables that are tied to the subcategories.

The risk prediction models can be dynamically updated by predictive modeling techniques that are configured to optimize the health outcome evaluation based on updated information, as described in further detail below. In some implementations, the risk models can be trained using historical data characterizing attributes of the clinical patient profile and/or the health outcome evaluation which is associated with overall risk profiles and with various risk subcategories, such as clinical, social, and behavioral risk factors. Some implementations of the current subject matter are able to assess and predict the overall risk of any patient, and categorize the sources of that risk into the aforementioned clinical, social, and behavioral risk subcategories.

At 140, treatment recommendations for the patient can be determined based on the calculated risk. In some implementations, the treatment recommendation can include data characterizing patient education materials, a medication action plan, and a medication list.

In some implementations, the treatment recommendation algorithm can provide a treatment recommendation that is based on each of the aforementioned risk components. For example, the social risk component, the behavioral risk component, and the clinical risk component of the determined risk predictions for the patient can each be used, either independently or in conjunction with one another, to determine a tailored treatment recommendation that minimizes each risk component. For instance, when the determined risk prediction for a patient indicates a high social risk, the determined treatment recommendation for this patient can include a referral to a social worker to learn about available resources to overcome access to care barriers. And, for instance, when the determined risk indicates a low social risk, the determined treatment recommendation for the patient would not include any suggested interventions to address access to care barriers.

In some implementations, the treatment recommendations can be determined based on the health outcome evaluation and/or received healthcare information data that is incorporated into the determined health outcome evaluation. The treatment recommendations can be generated by a recommendation rules engine.

In some implementations, when patients can have the same overall risk and risk components, the main drivers of the risk components (e.g., the various predictor variables) can be different, and the treatment recommendation rules engine can account for this variance in risk components in determining the specific treatment recommendations. The treatment recommendation rules engine with treatment recommendations will consider the weight of these risk components and the predictor variables within each risk component. For example, for one of the patients, if one of the significant predictor variables for behavioral risk is medication nonadherence for specific medications, the treatment recommendation algorithms will determine that one of the recommendations for this patient will be to perform adherence counseling for that particular medication. If the other patient, who has the same behavioral risk, does not have nonadherence as a significant variable and instead has a high clinical risk component instead, the treatment recommendation algorithm will determine a treatment recommendation to escalate therapy instead of adherence counseling.

In some implementations, the recommendation rules engine can include a rule execution engine that executes logic (e.g., one or more rules) to generate the treatment recommendation. For example, the rule execution engine of the recommendation rules engine can analyze inputs that can include the metrics/data outputs generated as part of the health outcome evaluation, the aforementioned risk predictions, data characterizing the patient’s health plan, data characterizing clinical guidelines, and/or other patient/provider healthcare data, and query a library of recommendation rules to obtain from the library the rules for execution that are relevant to the analyzed inputs, and execute the rules on the inputs to determine a treatment recommendation.

In some implementations, the recommendation rules engine can also include a verbiage retrieval process that can query a template database storing a variety of string templates for presenting the treatment recommendation. The verbiage retrieval can obtain from the template database an appropriate template based on the treatment recommendation. The recommendation rules engine can then generate a recommendation string based on the obtained template and that characterizes the treatment recommendation for providing to a patient and/or a provider, as described in further detail below. In some implementations, the verbiage retrieval process of the recommendation rules engine can translate the retrieved template based on the analysis of the inputs described above that is performed by the rule execution engine into an optimized recommendation string that is based on health outcome evaluation, the aforementioned risk predictions, data characterizing the patient’s health plan, data characterizing clinical guidelines, and/or other patient/provider healthcare data.

In some implementations, to determine the treatment recommendation, the recommendation rules engine can include a rule interpreter that can translate a high-level language into a complex set of rules for analysis on the inputs described above. Such functionality can allow for the complex set of rules to be determined without receiving data that characterizes the data structure of the analyzed inputs described above, which can allow for faster and more computationally efficient development of the rules used by the rule execution engine and expansion of the recommendation rules library.

In some implementations, to determine the treatment recommendation, the recommendation rules engine can include a rule interpreter that can translate a high-level language into a complex set of rules for analysis on the inputs described above. Such functionality can allow for the complex set of rules to be determined without underlying knowledge of the data structure of the analyzed inputs described above, which can allow for faster and more computationally efficient development of the rules used by the rule execution engine and expansion of the recommendation rules library.

In some implementations, the treatment recommendation can be determined based on the answers to the questions of the questionnaire data described above. For example, if the answers from the questionnaire indicate that a reason for patient nonadherence to a prescribed treatment plan is that the prescribed treatment causes undesirable side effects, the one or more treatment recommendation algorithms will determine a recommendation for an alternative treatment that does not cause these side effects based on an assessment of received data characterizing expert clinical knowledge, existing clinical guidelines and other third party data sources.

In some implementations, non-clinical recommendations beyond treatment recommendations can be determined. Such recommendations can include cost saving opportunities, medication switching opportunities to better align with financial incentives, social or behavioral care support programs, other programs patients may be eligible for either through their insurance or the government, or community-based resources that are available to the patient that can improve their health. These recommendations can be determined by a non-clinical recommendation algorithm that is substantially similar in operation to the treatment recommendation algorithm described above, but instead provides the aforementioned ancillary recommendations instead of treatment recommendations. The non-clinical recommendation algorithm can be trained based on data characterizing the opportunities available to the patient for a variety of patients having risk and demographic profiles with similarities to the determined risk prediction for the patient and the clinical patient profile, and the training of the algorithm can be routinely updated based on changes to the available opportunities.

In some implementations, the treatment recommendation can include a provider recommendation. The provider recommendation can include data characterizing treatment recommendations that are intended for use by the provider. The recommendations for providers can be determined in substantially the same way as the treatment recommendations for patients are determined. However, the provider recommendation algorithm can also utilize provider characteristics (demographics, specialty, prescribing patterns, site of practice) and clinical decisions made by the providers (which may or not be based on prior provider treatment recommendations) that are characterized by the aforementioned provider profile in generating the provider recommendations. In some implementations, the provider recommendation can include a listing of all treatment concerns identified and their suggested resolutions. The provider recommendation can also include data characterizing additional contextual information, such as critical patient data (e.g., recent hospitalizations), treatment guidelines sourced from clinical information reference databases, and an explanatory rationale for the recommendation that is generated by the recommendation rules engine. In some implementations, as explained in further detail below, the treatment recommendation rules engine can be trained and/or optimized based on historical data characterizing the relative success of recommended treatments for a variety of patients having risk profiles with similarities to the determined risk prediction for the patient.

At 150, the treatment recommendation can be provided. For example, in some implementations, wherein a provider recommendation is determined, a provider can review the provider recommendation in a user interface provided in a web page on a web browser of a client device of the patient that can provide all major issues identified and their suggested resolutions. For example, in some implementations, the treatment recommendation and/or the non-clinical recommendation can be provided to a user interface provided in a web page on a web browser of a client device of the patient, and the patient can accordingly view the treatment and/or non-clinical recommendations on their client device. For example, in some implementations, the treatment recommendations, which can include one or more of the aforementioned treatment recommendation, and the non-clinical recommendation, can be placed into a formatted material suitable for viewing by a patient and/or a provider. This material can be emailed, printed and mailed, and/or faxed. In some implementations, the provider treatment recommendation can also be pushed into electronic medical record systems. In some implementations, the user interface can generate and provide provider-level reports that feature several different provider treatment recommendations in order to advise a provider with respect to all patients needing care adjustments. In some implementations, additional and/or alternative recommendation documents or care plans can be provided to patients and providers based on the treatment recommendation, the non-clinical recommendation, and/or the provider treatment recommendation.

In some implementations, the suggested treatment recommendations can drive the creation of a task associated with the patient. These tasks can in turn be prioritized in a list format, such that platform users can view suggested actions aimed at reducing the patient risk. Each recommendation can include data characterizing an associated priority level and timeframe for action. For example, patients that have higher social, clinical and behavioral risk as determined by the models and recommendations for each patient, will have higher priority tasks than those with lower clinical social and behavioral risk or less weighted recommendations. In some implementations, a configurable workflow engine for executing one or more of the processes described herein can be included such that the assignment of tasks to care team members can align with each unique workflow and care team composition. The workflow engine can also pull data from the health outcome evaluation. The workflow engine can also analyze the history of interventions to determine the next course of action and priority level. For example, a task can be created to follow-up with the provider if they did not respond to the treatment recommendation as evidenced by a lack of medication change in the health outcome evaluation data.

In some implementations, the implementation of the suggested treatment recommendations by a patient and/or a provider can be continuously measured, recorded, and provided for use in future, iterative determinations of one or more of the aforementioned health outcome evaluation, the clinical patient profile, the provider profile, the risk prediction, and/or the treatment recommendations. In some implementations, the impact of the provided treatment recommendations can be quantified from both a clinical and economic perspective. For example, if the treatment recommendation indicates a patient be prescribed a statin for their diabetes, and data received by the system includes a pharmacy claims file that shows a statin has been prescribed and started, the system can mark the treatment recommendation as implemented. The clinical (e.g., lab values, improvement in health, etc.) and economic (e.g., total cost of care pre/post prescription of statin) impact of the recommendation being implemented can also be measured. In some implementations, the system can analyze the responses of the tailored questions across patients and the resulting clinical decisions, determine which questions lead to optimal interventions, and prioritize those questions for inclusion in the previously-described questionnaire data.

In some implementations, following completion of the treatment plan review by a provider and indication, by the provider, as to whether the treatment recommendation is to be implemented, ongoing changes in a patient’s health status can be detected via analyzing healthcare information data contained in data streams received from patient devices, provider devices, and/or external databases. For example, changes in medications (e.g., New medications added, dosage changes, medication discontinuation) can be monitored and used as a basis of determination of whether the recommendations were implemented. In some implementations, events, including but not limited to new medical diagnoses, new medications prescribed, new lab values, new device information, hospitalizations and emergency room admissions, provider visits can be identified, and a notification indicative of such an event can be pushed directly to a provider for further follow-up with the patient by the provider. This can prevent exacerbation of potential issues that could drive increased healthcare service utilization including but not limited to inpatient and emergency department visits, and ensure best-practice adherence. These flagged events also are data parameters considered in the questionnaire rules engine, workflow rules engine, and recommendation rules engine, as well as predictive models for use in the determinations described elsewhere herein.

In some implementations, when additional data is received that can be used to relate patient behaviors to clinical, social, and economic outcomes, predictive algorithms can use the received healthcare information data (which, in some implementations, can also include data characterizing feedback on interventions implemented that are based on and/or suggested by the provided treatment recommendations, data characterizing a decision and/or rationale not to implement any interventions that are based on and/or suggested by the provided treatment recommendations, and data characterizing the efficacy of the interventions implemented based on the provided treatment recommendations) and/or determined health outcome evaluations that are based on data characterizing feedback on interventions implemented that are based on and/or suggested by the provided treatment recommendations, data characterizing a decision not to implement any interventions that are based on and/or suggested by the provided treatment recommendations, and data characterizing the efficacy of the interventions implemented based on the provided treatment recommendations to further tailor patient recommendations, provider recommendations, tasks and interventions by updating the various rules engines described in detail above. In this way, maximal impact on clinical outcomes and patient quality of care can be achieved. The various rules engines and algorithms described elsewhere in detail herein can be updated by use of a predictive model that identifies patterns in adherence to interventions suggested by the treatment recommendation by analyzing data characterizing healthcare benefits, data characterizing past patterns of adherence to treatments prior to any interventions suggested by the determined treatment recommendations, patient demographic data, patient geospatial data, data characterizing healthcare utilization and spending patterns, data characterizing medication utilization patterns, patient-reported data, and/or results from other predictive models described elsewhere herein. The predictive model can identify, via this analysis, those interventions that result in improved adherence to interventions and determine predictor variables that can be added to the rules stored in the various rules libraries described elsewhere herein and/or modifications to one or more of the rules stored in the rules libraries described elsewhere herein that can result in rules engines outputs that are more accurate and more likely to be predictive. For example, the predictive model can add predictor variables to the rules stored in the recommendation rules library that can cause the recommendation rules engine to determine treatment recommendations that are more likely to cause improved interventions utilized by providers and/or patients and thereby cause improved health outcomes. For example, the recommendations in the recommendation rules engine can be initially based on treatment guideline parameters, and can than become more targeted over time as more data is considered in the predictive models. Data used to create guidelines are typically from randomized clinical trials based on smaller sample size, homogeneous population, limited number of data elements collected. This can lead to predictors being limited to age, race, gender, drug groups, disease groups. Moreover, in the guidelines, these predictors can then be turned into binary such as age > 65 or < 65. If a patient has uncontrolled type 2 diabetes and concurrent cardiovascular disease, the initial recommendation is to recommend a glucagon-like peptide-1 agonist or sodium-glucose co-transporter-2 inhibitors per the American Diabetes Association. For example, the machine learning models identify other data predictors of treatment success (in this case defined as diabetes control via measurement of hemoglobin A1c), such as another co-morbid condition or specific demographics such as age range, this comorbid condition and age range can be added to the logic of the recommendation rules engine to incorporate parameters beyond those considered in treatment guidelines.

For example, in some implementations, the system can predict an optimized treatment intervention and provide a recommendation for implementing the optimized treatment intervention to a patient and/or provider. Based on feedback indicating that the intervention was successful, the system can identify a predictor of success using a machine learning model. For example, the machine learning model can identify the predictors of diabetes control via measurement of hemoglobin A1c as an indicator of treatment success. Using the identified predictors of success, the system can generate additional logic (e.g., modifications to a rule, a new rule, a deletion of a rule, and the like) that can be inserted into the recommendation rules engine. For example, the system can generate a rule indicating that a predetermined age range is a predictor of success with a given treatment/intervention recommendation should be provided in response to determining that a patient is within a predetermined age threshold. For subsequent recommendations, the modified recommendation rules engine will implement the logic indicating that a predetermined age range is a strong predictor of a successful intervention and thereby provide an improved recommendation for the intervention.

For example, a predictive model for medication adherence can be used for prediction whether a patient is likely to remain adherent or become non-adherent to a drug. The predictive model can evaluate patient demographic and socioeconomic data, the name of the medication, diagnoses, any patient comorbidities, any side effects reported by the patient and other relevant information including historical behaviors. The predictive model can analyze this data not just for the particular patient, but also many other patients determined to have similar profiles and characteristics based on the data inputs and parameters. In some implementations, the predictive model can perform this analysis by assessing this data and determining predictive variables that are significant predictors of a successful treatment outcome. The determined predictive variables can be applied to data characterizing a broader patient population to identify other patients who have a high likelihood of achieving a successful treatment outcome. Based on this analysis, and the historical performance of medication adherence for all patients with similar profiles and characteristics, the algorithm can predict the likelihood of a patient becoming non-adherent to their medications, and the expected timing of the start of that non-adherence event.

In addition, in some implementations, the aforementioned risk prediction parameters can be assessed on a continuous basis to create a dynamic risk score made up of a composite of all these parameters, related to how they affect a patient’s health and wellbeing. The software platform can be configured to prompt an intervention (e.g., by creating a “just in time” recommendation) before the patient becomes non-adherent. These models can rely on a feedback loop based on the success or failure of previous and similar interventions and/or recommendation timings to further hone the timing and type of intervention required to successfully address non-adherence.

In some implementations, the treatment recommendation rules engine can be modified based on predictive modeling techniques that can predict methods of interaction with patients and providers that are most likely to result in improved health outcomes. For example, the system can analyze provider characteristics such as location, specialty, prescribing history, preferred treatment plans, and the like, and can compare the provider to other similar providers. The software platform can then determine an optimal timing (e.g., Monday mornings), format (e.g., fax to office), and frequency (e.g., once a week) for delivering the treatment recommendations, in order to maximize the chance of the recommendations being implemented based on the provider’s characteristics and behavior. These features can be implanted based on a feedback loop of data including the success of past interventions in order to further determine the optimal timing and type of recommendation delivery required to ensure implementation by providers. The outcomes, such as clinical response and success of implementation of interventions, are tracked and the impact of the interventions are labeled and fed back to the aforementioned models/rules engines for inclusion as additional features in the models. In the next iteration of the model, these new significant features will be incorporated in the models to further refine risk predictions and future interventions.

In some implementations, the platform can also automate outcome reporting and can provide continuous visibility and transparency into patient and provider performance and success. Outcomes measured and included as part of the outcome reporting can include changes in medication adherence, total cost of care, total medical costs, total pharmacy costs, number of emergency department visits and hospitalizations, percentage of medication errors resolved, engagement measures related to patient, provider and pharmacy outreach, and whether providers have implemented the suggested recommendations.

The platform can additionally measure outcomes such as patient satisfaction, provider satisfaction, time to implementation of a recommendation, best time of day to deliver a recommendation, best method to deliver a recommendation (such as but not limited to telephonically, via email, via electronic health record messaging, via fax), patient and provider engagement. Other measured outcomes calculations are referred to elsewhere above. (e.g., visits, hospitalizations, % of errors resolved etc.) Engagement measures such as telephone pick up rate, fax receipt rate etc. can be measured via logging of phone calls and their associated outcomes either automatically (e.g., a phone call is picked up, length of call, time of day of call, timezone of recipient etc.) or manually via the platform’s logging function. Patient/provider satisfaction can be measured via a questionnaire administered directly from the portal, whereby a patient/provider answer is recorded in the platform and aggregated with other patient/provider answers as part of a data collection effort to understand satisfaction and produce a satisfaction score based on the stakeholder. Time to implement a recommendation can be calculated as the time from when a recommendation is sent via fax, email or telephonically delivered to the prescriber, and when the prescriber implements the change, viewed as either from the data feed (e.g. adding a medication, the date the new medication was added based on the recommendation) or faxed/phone receipt of recommendation implementation as provided directly by the provider.

In some implementations, this functionality can be implemented by the use of an automated outcomes analysis engine, which can include one or more of the following components: an extract-transform-load (ETL) process, an analysis planner, a report planner, an analysis plan executor, and a report generator. The ETL process can include data loading from multiple data sources, data extraction and normalization, aggregation and storage of the data in a data warehouse. The analysis planner can include an analysis plan loader that includes a list of analyses to be executed, execution schedule, cohort definition and parameters for each analysis. The report planner can include a reporting plan that includes a list of analyses to be included a report, report templates, delivery methods and recipients. The analysis plan executor can include a data analysis pipeline that can load a data analysis plan from the data warehouse and executes the analyses scheduled by the analysis plan, and results from each analysis can be stored in the data warehouse. The report generator can monitor the analysis results of an analysis plan. When results become available in the data warehouse, the report generator can generate reports and deliver them according to the report plan. Multiple report plans can be created for the same or a subset of analysis results. This includes different visualization templates, delivery methods or recipients.

These measurements and member, provider and clinical content attributes can be used to automatically adjust the weighting of parameters used by the aforementioned rules engines and algorithms in an automatic way. The observation of outcomes associated with 1) the content of the rules engines/algorithm 2) the method of delivery of the clinical recommendations 3) the attributes and content related to the patient that can be used by the rules engines/algorithms, including the risk profiling described elsewhere herein 4) the attributes and content related to the provider that can be used by the rules engines/algorithms, and 5) the impact on clinical and economic outcomes described elsewhere herein can be used to inform the subsequent iterations of the algorithm and recommendation. This can ensure that relative probability and weighting of each unique data label or feature is taken into account for subsequent algorithms and recommendations that can be delivered. Based on this automated measurement and recalibration of algorithms, each recommendation delivered will, over time, become more impactful and more likely to drive target results.

For example, the aforementioned algorithms/rules engines can identify a patient with high behavioral risk and recommend to a provider, via a user interface, to start this patient on an antipsychotic medication for an untreated bipolar disorder. The system can track whether the medication is added to the patient’s medication regimen via the algorithmic review of the patient’s data. Once prescribed, the platform analyzes the patient’s risk profile as described elsewhere herein, alerting the provider via the user interface that the patient remains at high behavioral risk due to nonadherence, also identified from the patient data. This prompts the generation of a tailored questionnaire as described elsewhere herein to prompt the user to identify the reasons for nonadherence. The patient answers to the tailored questionnaire can be analyzed to determine that a likely cause of the patient’s nonadherence is due to a side effect of weight gain. The platform can analyze the new set of patient data, and can determine a treatment recommendation for the patient for an alternative medication that does not have the same degree of metabolic adverse effects. The provider can deliver this treatment recommendation, and the system can monitor whether the medication is added to the patient regimen. Once the medication is added, the patient’s behavioral health risk is deemed by the system to decrease and the member is stabilized. All of these data points can be fed back into the rules engines/algorithms and, as the rules engines/algorithms learns and observe these patterns across patients, the system can then recommend alternatives for other patients who have similar social, clinical and behavioral characteristics and risk factors to proactively prevent nonadherence to this particular antipsychotic and prevent rising behavioral health risk.

In some implementations, the system can include one or more modules corresponding to more specific medication-related issues such as, adherence to quality measures, drug product selection, drug dosing regimen, drug-drug interactions, drug-disease interactions, adverse effects/events, contraindications, patient product misuse, formulary switching opportunities, adherence to treatment guidelines, adherence to chronic medications, patient education needs, social assistance needs, required lab tests, required health screenings, required medical appointments, required pharmacy appointments, disease management needs, utilization needs. Each module can be customized to meet the most pressing needs of the system users, such as specific programs, health conditions, disease areas, medication classes, therapeutic areas, formularies, or education issues that are of particular focus. Although a few variations have been described in detail above, other modifications or additions are possible. For example, some implementations of the current subject matter can be used to optimize the dose of a patient’s medications based on their clinical reaction to the medication and the results that medication is achieving. For example, a patient currently prescribed a low dose of statin, who is adherent to their medication, but continues to display high cholesterol levels may receive a recommendation to increase their dose to a higher level or switch to a moderate or high-intensity statin based on their clinical profile.

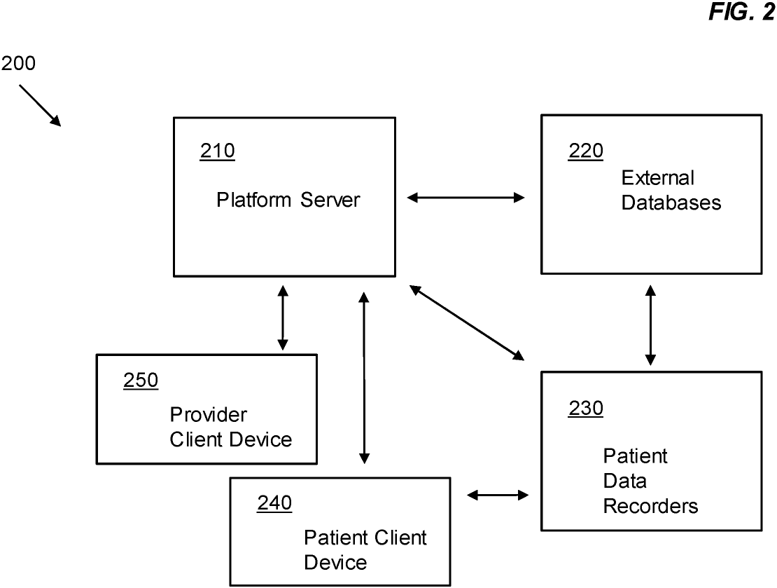

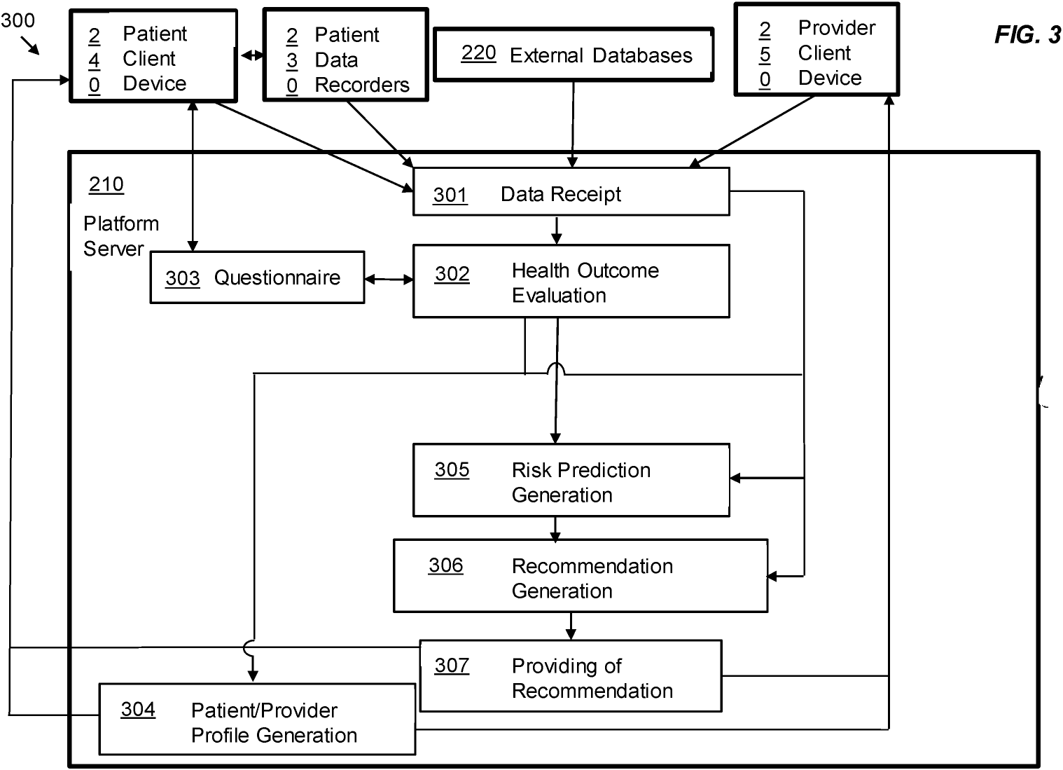

FIG. 2 is a system diagram 200 illustrating an example system of some implementations of the current subject matter that can provide for the functionality described herein, and FIG. 3 is a data flow diagram 300 illustrating the transfer of one or more of the types of data described herein between the system components illustrated in FIG. 2 and in accordance with some implementations of the current subject matter. As shown in FIG. 2, the system 200 can include a platform server 210 that is configured to perform one or more of the processes described herein. For example, the platform server 210 can receive data from a variety of sources, such as various external databases 220 that house healthcare information data, patient data recording devices 230 that are configured to record physiological parameters and/or biomarkers of a patient, a client device 240 (e.g., mobile device, personal computer, etc.) of a patient that is configured to receive inputs from the patient that pertain to the applicable patient-related data forms described elsewhere herein, and a client device 250 (e.g., mobile device, personal computer, etc.) of a provider that is configured to receive inputs from the provider that pertain to the applicable provider-related data forms described elsewhere herein.

As shown in FIG. 3, healthcare information data as described elsewhere herein (and feedback data characterizing interventions made in the care of a patient and other forms of feedback from prior outputs of the platform server 210 and processes described elsewhere herein) can be received by the platform server 210 in a data receipt process 301 from one or more of the various external databases 220, the patient data recorders 230, the patient client device 240, and/or the provider client device 250. The data received at data receipt process 301 can be provided to a health outcome evaluation process 302, which executes various processes described elsewhere herein to determine the health outcome evaluation. In some implementations, wherein it is determined during the health outcome evaluation process 302 that the patient needs to answer some questions to address deficiencies in the received data in order for the health outcome evaluation process 302 to fully formulate the health outcome evaluation, the health outcome evaluation process 302 can generate applicable questions to address the deficiencies and provide them to the questionnaire process 303. The questionnaire process 303 can incorporate the questions into questionnaire data that characterizes the questions. Once that is complete, the questionnaire data can be provided to the patient client device 240 for display on a graphical user interface of the patient client device 240. The patient can answer the questions by interacting with the graphical user interface of the patient client device 240, and data characterizing the answers can be provided by the patient client device 240 to the platform server 210 as an input to the questionnaire process 303. The answers can then be extracted from the answer data provided by the patient client device 240 and passed back to the health outcome evaluation process 302 for completion of the determination of the health outcome evaluation.

The health outcome evaluation, which can be an output of the health outcome evaluation process 303, can be provided to the patient/provider profile generation process 304, which can generate one or more of the patient and/or provider profiles described elsewhere herein in accordance with the processes described elsewhere herein based on the health outcome evaluation and/or the data received by the platform server 210 at data receipt process 301 (which can also be provided to the patient/provider profile generation process 304. The patient and/or provider profile can be output from the patient/provider profile generation process 304 and provided for display on the patient client device 240 and/or the provider client device 250 for depiction thereon. And, the health outcome evaluation can be provided to the risk prediction generation process 305, which can generate the risk predictions described elsewhere herein based on the health outcome evaluation and/or the data received by the platform server 210 at data receipt process 301 (which can also be provided to the risk prediction generation process 305). The risk prediction generation process 305 can output data characterizing the generated risk predictions to the recommendation generation process 306, which can generate one or more of the recommendations described elsewhere herein based on the received risk predictions and/or the data received by the platform server 210 at data receipt process 301 (which can also be provided to the recommendation generation process 306). The generated one or more recommendations can be provided to the recommendation providing process 307, which can format the one or more recommendations in accordance with the techniques described elsewhere herein and provide the formatted recommendation to the patient client device 240 and/or the provider client device 250.

The subject matter described herein provides many technical advantages. For example, some implementations of the current subject matter can allow for real-time updating of the treatment plan changes and recommendations during the conversation with the patient or the provider. If the user of the software learns new information in the course of patient care or a conversation with the patient or provider, they can add the data to the patient or provider profile and analyze the additional data in context in real time to update the treatment profile. Additionally, the system, by performing the operations described in detail herein can rank and prioritize patients and providers most in need of intervention based on their clinical and economic risk, thus allowing users to most efficiently tackle the population and address gaps in care, delivering recommendations to those patients and providers who would most benefit from the intervention.

By generating the aforementioned recommendations and automatically generating the patient and provider materials, which may be based on the recommendations, which can be delivered electronically, via facsimile, or can be printed for mailing, the clinician can operate 5-10× more efficiently and can focus on clinical counseling. By having the software platform detect existing issues and provide the associated recommendations, pharmacists, nurses, and other qualified healthcare professionals can be empowered to work at the top of their license as trained medication specialists. Now, during counseling sessions with patients, the qualified healthcare professionals can focus on medication education and counseling instead of documentation and investigation. This can decrease the time for a comprehensive medication review with a patient from 1 hour to 5-10 minutes. The platform is also built according to Fast Healthcare Interoperability Resources (FHIR) standard for health data exchange to allow communication with providers via integration with other electronic health records and software systems. The treatment recommendations can be automatically generated and can be reviewed by licensed clinical personnel and delivered to the patients and their care teams electronically or physically without the need for significant data entry or manual labor, increasing the efficiency of the clinical personnel, often pharmacists, by 5-10×. The treatment recommendations can also be delivered to the patients and their caregivers directly.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

Certain exemplary embodiments are described herein to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon.

What is claimed is:

1. A method comprising:
receiving data characterizing healthcare information associated with a patient;
determining a health outcome evaluation for the patient based on the received healthcare information data;
determining a risk prediction for the patient based on the determined health outcome evaluation;
determining a treatment recommendation for the patient based on the determined risk prediction; and
providing the treatment recommendation.

2. The method of claim 1, wherein the determining of the health outcome evaluation includes:
comparing the received healthcare information data to healthcare data characterizing a predetermined set of healthcare parameters for an aggregated population of patients,
determining a deficiency in the received healthcare information data based on the predetermined set of healthcare parameters,
generating questionnaire data that characterizes at least one question based on the determined deficiency,
providing the questionnaire data to a client device of the patient, and
receiving, from a client device, answer data characterizing at least one answer to the at least one question characterized by the questionnaire data;
and wherein the health outcome evaluation is based on the answer data.

3. The method of claim 2, wherein the generating of the questionnaire data includes:
querying a questionnaire rules engine for the at least one question based on the determined deficiency, the questionnaire rules engine configured to generate the at least one question, wherein the questionnaire rules engine is modified by a questionnaire predictive model that identifies a predictor variable based on the received healthcare information data and revises the questionnaire rules engine based on the identified predictor variables, and
receiving the at least one question from the questionnaire rules engine for inclusion in the questionnaire data.

4. The method of claim 1, further comprising:
determining a clinical patient profile for the patient based on the received healthcare information data and the determined health outcome evaluation, the clinical patient profile characterizing an attribute of the patient.

5. The method of claim 1, further comprising:
determining a provider profile for a provider of healthcare services to the patient based on the received healthcare data, the provider profile characterizing an attribute of the provider.

6. The method of claim 1, wherein the determining of the risk prediction for the patient includes executing a risk prediction model for a risk factor that predicts a likelihood of a negative health outcome, the risk prediction model trained for providing the risk factor in response to the querying based on historical patient risk data.

7. The method of claim 6, wherein the determining of the treatment recommendation includes:
querying a treatment recommendation rules engine for a recommendation parameter based on at least one of the determined risk factor, the health outcome evaluation, and/or the received healthcare information data, the querying including execution of a recommendation rule by the treatment recommendation rules engine, and
generating a recommendation string that characterizes the recommendation parameter.

8. The method of claim 7, wherein the treatment recommendation rules engine is modified by a predictive model that identifies a predictor variable characterizing a likelihood of success of an intervention characterized by the treatment recommendation, the identifying based on received feedback data that indicates a level of success of the intervention, determines a modification to the recommendation rule based on the identified predictor variable, and modifies the recommendation rule based on the determined modification.

9. The method of claim 7, wherein the providing of the treatment recommendation includes transmitting the recommendation string for presentation on a graphical user interface of a client device.

10. The method of claim 7, wherein the treatment recommendation rules engine is modified by a recommendation predictive model that identifies a predictor variable characterizing a pattern in adherence to interventions suggested by the treatment recommendation based on the received healthcare information data and modifies a rule of the treatment recommendation rules engine based on the identification.

11. The method of claim 6, wherein the determining of the risk prediction for the patient includes determining a clinical risk parameter characterizing a level of clinical risk based on the determined health outcome evaluation, determining a social risk parameter characterizing a level of social risk based on the determined health outcome evaluation, and determining a behavioral risk parameter characterizing a level of behavioral risk based on the determined health outcome evaluation.

12. The method of claim 11, wherein one or more of the clinical risk parameter, the social risk parameter, and the behavioral risk parameter is dynamically updated based on received feedback data characterizing the patient.

13. A system comprising:
at least one data processor; and
memory storing instructions configured to cause the at least one data processor to perform operations comprising:
receiving data characterizing healthcare information associated with a patient;
determining a health outcome evaluation for the patient based on the received healthcare information data;
determining a risk prediction for the patient based on the determined health outcome evaluation;
determining a treatment recommendation for the patient based on the determined risk prediction; and
providing the treatment recommendation.

14. The system of claim 13, wherein the determining of the health outcome evaluation includes:
comparing the received healthcare information data to healthcare data characterizing a predetermined set of healthcare parameters for an aggregated population of patients,
determining a deficiency in the received healthcare information data based on the predetermined set of healthcare parameters,
generating questionnaire data that characterizes at least one question based on the determined deficiency,
providing the questionnaire data to a client device of the patient, and
receiving, from a client device, answer data characterizing at least one answer to the at least one question characterized by the questionnaire data;
and wherein the health outcome evaluation is based on the answer data.

15. The system of claim 14, wherein the generating of the questionnaire data includes:
querying a questionnaire rules engine for the at least one question based on the determined deficiency, the questionnaire rules engine configured to generate the at least one question, wherein the questionnaire rules engine is modified by a questionnaire predictive model that identifies a predictor variable based on the received healthcare information data and revises the questionnaire rules engine based on the identified predictor variables, and
receiving the at least one question from the questionnaire rules engine for inclusion in the questionnaire data.

16. The system of claim 13, wherein the determining of the risk prediction for the patient includes executing a risk prediction model for a risk factor that predicts a likelihood of a negative health outcome, the risk prediction model trained for providing the risk factor in response to the querying based on historical patient risk data.

17. The system of claim 13, wherein the determining of the treatment recommendation includes:
querying a treatment recommendation rules engine for a recommendation parameter based on at least one of the determined risk factor, the health outcome evaluation, and/or the received healthcare information data, the querying including execution of a recommendation rule by the treatment recommendation rules engine, and
generating a recommendation string that characterizes the recommendation parameter.

18. The system of claim 17, wherein the treatment recommendation rules engine is modified by a predictive model that identifies a predictor variable characterizing a likelihood of success of an intervention characterized by the treatment recommendation, the identifying based on received feedback data that indicates a level of success of the intervention, determines a modification to the recommendation rule based on the identified predictor variable, and modifies the recommendation rule based on the determined modification.

19. The system of claim 17, wherein the treatment recommendation rules engine is modified by a recommendation predictive model that identifies a predictor variable characterizing a pattern in adherence to interventions suggested by the treatment recommendation based on the received healthcare information data and modifies a rule of the treatment recommendation rules engine based on the identification.

20. A non-transitory computer program product storing instructions which, when executed by at least one data processor forming part of at least one computing system, cause the at least one data processor to implement operations comprising:
receiving data characterizing healthcare information associated with a patient;
determining a health outcome evaluation for the patient based on the received healthcare information data;
determining a risk prediction for the patient based on the determined health outcome evaluation;
determining a treatment recommendation for the patient based on the determined risk prediction; and
providing the treatment recommendation.
